(19) United States
(12) Patent Application Publication
(10) Pub. No.: US 2010/0071429 A1
(43) Pub. Date: Mar. 25, 2010

(54) METHOD AND DEVICE FOR THE TREATMENT OF ORGANIC RESIDUAL PRODUCTS OF BIOGAS SYSTEMS

(76) Inventor: Reinhart Von Nordenskjöld, Egmating (DE)

Correspondence Address: CHERNOFF, VILHAUER, MCCLUNG & STENZEL, LLP, 601 SW Second Avenue, Suite 1600, Portland, OR, 97204, US

(21) Appl. No.: 12/522,144

(22) PCT Filed: Jan. 4, 2008
(86) PCT No.: PCT/DE2008/000007

§ 371 Date: Jul. 2, 2009

(30) Foreign Application Priority Data
Jan. 5, 2007 (DE) 10 2007 001 177.8
Jan. 31, 2007 (DE) 10 2007 004 892.2

(51) Int. Cl. C05F 7/00; C12M 1/00

(52) U.S. Cl. 71/11; 435/290.4

(57) ABSTRACT

The invention relates to a method and a device for the conditioning of organic residuals occurring, for example, in biogas systems, wherein a digestate materializes from the organic residuals as an intermediate product during biogas regeneration in a fermenter, the digestate being subjected to mechanical conditioning comprising at least one concentration step. Subsequent to the mechanical conditioning, a first part of the liquid digestate passes through an evaporation process for the production of a top dressing, and a second part of the concentrated digestate passes through a drying process for the production of base dressing, wherein a mass flow of the top dressing concentrate is fed to the partial flow of the base dressing production such that the base dressing is enriched upgraded. Due to this enrichment, the digestate can either be gasified for energy generation, or utilized as an organic agricultural fertilizer.

### BACKGROUND OF THE INVENTION

The invention relates to a method and a device for the treatment of organic bio-residuals, particularly domestic and/or commercial residuals, including raw and/or cooked waste food, agricultural residuals, particularly animal excrement and/or plant waste, but also plant substances cultured especially for the process, in which these substances are supplied in part to an anaerobic reactor and in which the biogas resulting from fermentation is drawn off and the remaining digestate supplied for further usage.

For digesting solid and liquid organic biomass it is known from prior art to acidify a crude fraction—where necessary following mechanical treatment (for example sand trap)—and forward it to fermentation to generate biogas. In addition to producing the actual biogas this so-called methanization also results in a digestate residual fraction which can be discharged as compost or further processed into a fertilizer additive. This residual fraction has a dry substance (DS) content of approximately 6-7%. Such a digestion method can be put to use in all for liquid manure, maize silage, cereals or whey or similar substances. However, it is not every starting biomass that is equally suitable for fermentation, there often being a problem of non-fermentable residuals remaining which need to be disposed of or recycled separately. Inadequately fermentable are especially residuals containing lignin or the like. These residuals are often processed further into fertilizer dressings.

In general, an organic residual having a minimum content of >3% of nitrogen or phosphorous or potassium in relation to the dry substance fraction is defined a “dressing”. In processing the aforementioned digestate into a crude dressing the digestate after subsequent mechanical conditioning with separation can be forwarded for concentration in two partial flows. Whilst the first partial flow is subjected to drying to produce a dry, so-called base dressing having a dry substance fraction of around 85%, the second partial flow of the liquid fraction remaining after compaction is put through evaporation to produce a top dressing with a dry substance fraction of approximately 15%. Both crudes can be recycled agriculturally when their nutritional content is high enough—the base dressing preferably in autumn to get plants in growth through the winter, and the top dressing preferably in spring. Although the resulting digestate, especially in largish systems of ≧1 MW, poses a disposal problem because of the quantity involved, its conventional use, irrespective of its composition, is in agriculture. However, although the wet top dressing stemming from evaporation of the digestate has a minimum 6 percent content of nitrogen as a rule, the 3 percent content of nitrogen as a rule in the dry base dressing stemming from drying the digestate is deficient. It will be appreciated that the fraction of these three dressing components nitrogen, potassium and phosphorous depends on the composition of the digestate residual and also on how well this digestate is capable of binding, for example, ammonia to the nitrogen contained therein.

The object of the invention is to define a method and a device of the aforementioned kind which now makes it possible to improve treatment of organic residuals to generate a recyclable product, such as, for example, a source of energy, but also a recyclable product in the form of fertilizer dressings as sources of energy. The method is also intended to optimize the energy cycle as regards processing regrowth crudes involving in detail multistage vacuum evaporation assignable conditioning without the need of outside energy.

### SUMMARY OF THE INVENTION

This object is achieved basically by the method as recited in claim 1. Advantageous aspects of the method read from the dependent sub-claims. The device in accordance with the invention reads from claim 10. Advantageous aspects of the device are recited in the dependent sub-claims.

The gist of the invention is to improve recycling energy from, for example, the digestate resulting from the organic residuals in generating biogas by recombining at least in part the digestate cake resulting from concentration and the liquid flow stemming therefrom after having subjected the liquid flow to a treatment for retention of a concentrate and a condensate from the liquid flow. Whilst the condensate consists near totally of water, the other components in the liquid flow are concentrated in the concentrate. With this concentrate as thus obtained, the digestate cake can be reenriched and subjected to further conditioning, for example, in the form of drying or pelletizing. The result of this is that then all components existing in the digestate are separated from the water fraction of the digestate, and these components then concentrated so that recycling the resulting base dressing concentrate furnishes an enhanced recovery in further utilization. The meaning of this is that the invention now makes it possible to optimally recycle the digestate resulting e.g. in generating biogas by upgrading it—where necessary—by the energy made available in generating the biogas in generating electricity. “Upgrading” in this sense is to be appreciated both as better handling by concentration—by a factor of e.g. 1 to 10—improved storageability, usefulness for generating heat or enrichment with additives as well as any other processing thereof, such as drying. With the aid of the method in accordance with the invention an ecological and thus sustainable method is now available for also recycling, for example, ligninated biomass.

In accordance with one advantageous embodiment use is made of the waste heat resulting in recycling the biogas for drying. If the system is intended to be operated totally self-supporting the available waste heat dictates the content of the water fraction of the base dressing mixture resulting from base dressing and the transposed concentrate. In other words, when sufficient heat is available the entirety of the recovered concentrate can be mixed into the concentrated cake and the mixture subsequently dried. It has found to be an advantage to feed the concentrate to the base dressing during its drying (as will be explained later in conjunction with a belt dryer).

In accordance with another advantageous embodiment the treatment for extracting a concentrate and a condensate from the liquid flow is done by means of vacuum evaporation. As an alternative it is just as possible to make this extraction by means of ultrafiltration with reverse osmosis, the resulting retentate corresponding to the concentrate enriched with the components of the digestate.

Advantageously, the mass flow of concentrate transposed into the base dressing is selected so that at least the enriched dry product, i.e. the base dressing after being dried has a minimum content of 3% nitrogen and/or potassium and/or phosphorous or compounds thereof. The resulting product has then the standing of an organic fertilizer.

Described in the following is the basic scheme of the method in accordance with the invention by way of a basic recycling mode solely relating to the production of organic fertilizers.

In the basic recycling mode of the invention converting deficient quality biomass into organic fertilizers is done by returning the excess nitrogen, potassium or phosphorous fractions resulting from processing the digestate, to the deficient quality dressing flow, i.e. mostly the base dressing in actual practice—to a degree in advantageously making available the necessary energy from the waste heat of the biogas thermal power plant. The transfer of this energy to drying or evaporation is done in practice as a rule by heat exchangers, by means of which the waste heat is output to the evaporators or dryers/conditioners. To meet this energy requirement the mass fraction of the wet top dressing to be fed to the base dressing for the drying is dictated by the amount of energy available from the waste heat of the biogas thermal power plant. The meaning of this is that the percentage of transposed highly nutritious top dressing to the base dressing originally lacking quality because of it having less than 3% potassium or phosphorous or nitrogen may be all the more, the higher the amount of energy from the waste heat of the biogas thermal power plant so that the enriched base dressing can still be adequately dried. Because of the waste heat energy as made available, the parameters concerning the extent of a) evaporation, b) drying and c) mass flow of transposed top dressing need to be selected such that the base dressing resulting from processing must have a content of at least 3% of potassium or phosphorous or nitrogen. Tests have shown that upgrading the base dressing to an organic fertilizer without the addition of dressings “from outside” is possible when using what is called a wet fermenter for biogas generation in which an excess amount of energy in the waste heat of the biogas thermal power plant for operating drying and evaporation such that from this excess energy a mass transfer from top dressing to base dressing can be realized to the extent of 20% of the top dressing produced in all.

The components of the crude digestate fluctuate in amount, causing also its technical properties for processing to vary. This influences extracting the solids which in turn affects the drying and evaporation performance and ultimately the amount of concentrate produced therefrom. Whilst the amount of concentrate of produced base dressing with maximum evaporation of the digestate is lowest, the amount of concentrate to be driven off is all the more, the higher the percentage of dry substance after concentration.

To continually maintain the production of organic fertilizer it is provided for in accordance with one advantageous embodiment to add an acid, for example H2SO4 to the concentrated condensate to bind the volatile fraction of nitrous compounds, such as ammonia in the digestate. This binding increases the percentage of nitrogen in the treated partial flows as a rule in both partial flows, i.e. in the production of dry base dressing and in the production of wet top dressing. In addition to this, binding these nitrous compounds in the digestate has the advantage that there is no stench nuisance as would be the case, for example, when gassing out ammonia from the digestate.

The advantage in accordance with the invention in adding sulfuric acid after having concentrated the crude digestate into the resulting digestate cake is that it inhibits frothing whilst also avoiding flotation since the solids exist in the compacted cake. Should acidifying the liquid fraction of the digestate result in frothing, anti-frothing agents or mechanical means of combating frothing can be provided. Intensive testing has proven that the residence time and temperature of the digestate plays an important role in inhibiting frothing. This is why residence tanks are interposed and/or the digestate preheated. To finish with, thermal insulation is provided for the concentrate to maintain the temperature with a minimum expense of energy in thus running the system with as much dry substances as possible.

In accordance with a further advantageous embodiment of the method in accordance with the invention mechanical conditioning involves, in addition to the aforementioned press or decanter concentration, sifting/fine sifting by means of, for example, a vibration sieve the liquid fraction materializing from concentration to filter out fibrous fines which could otherwise cause blockage when evaporating the top dressing in evaporators or in ultrafiltration/reverse osmosis.

It is considered an advantage to buffer the resulting digestate intermediate product from anaerobic methanization in a digestate buffer to save extra capacity in processing the digestate.

As regards resting storage it is furthermore an advantage to rest the substrate before acidification in the case of maize silage or other silages or fibrous substances for at least 0.5 h to allow it to age in getting rid of air inclusions in the substrate. In addition to inhibiting floating scum in the anaerobic reactor this is also of advantage in processing the digestate that ammonification (formation of NH4 from NH3) and acidification proceed with no problem, in other words enabling the pH to be tweaked more reliably and calculable when acidifying the digestate.

In mixing the controlled fraction of the mass flow of top dressing in the base dressing tests have shown it to be a major advantage to use a belt dryer for “raining” the partly dried base dressing with the top dressing by, for instance, a method in which the drying material leaving the conditioner is composed of two different wet fraction strands, namely on the one hand the base dressing and on the other the top dressing which differ by the dry substance content. These two wet fractions are brought together during drying in the drying area and obtained as the dried product. This novel concept of the conditioner and how it works in this respect now makes it possible to dry a wet fraction, namely the top dressing, which because of its low dry substance content can be considered to be a fluid, i.e. a practically liquid fraction. Drying a wet fraction having such a low dry substance content is done by first adding to the latter a wet fraction to be dried functioning as a vehicle for the “liquid” second wet fraction. The end product of the dryer has a dry substance content of approximately 80%. Due to adding the top dressing “enriched” with nitrogen, potassium or phosphorous the end product leaving the dryer has a minimum content of 3% of at least one of the these substances, i.e. this being a dressing which in addition is also purely organic. Enriching the dry base dressing into a full-value organic fertilizer has the advantage of causing a much less stench nuisance as compared to direct field spraying with liquid manure. Furthermore, all transport expenditures involved become less due to the lesser weight of a dry dressing as compared to the digestate substrate as the dressing.

For fermenting in generating biogas a wet fermenter can be employed which for good functioning requires a minimum water content, or, expressed otherwise, without exceeding a maximum dry substance content, roughly 10-13% dry mass being usual, depending on the crude involved. To ensure that the water requirement is not exceeded it can be provided for in accordance with another advantageous embodiment of the invention to return the condensate resulting from evaporation of the top dressing or drying the base dressing by directly apportioning it in mashing or, as an alternative, dosing it into the fermenter feeder. When these condensates contain froth or a lot of substances that can be evaporated, reverse osmosis can be employed so as not to unnecessarily load mashing but especially acidification and fermentation e.g. with solids and/or salts.

Irrespective of the energy required for returning the condensate to the fermenter it is to be emphasized that returning the condensate in the mash saves much more energy than in feeding fresh water from without the system, since the condensate already has an elevated temperature (approximately 55° C.) with which the temperature of the crude can be increased. In addition to this, use of the condensate in this way is an elegant and economic solution because any form of “discharge” may involve soilage.

Instead of a wet fermenter a dry fermenter can also be used. In a dry fermenter it is possible to methanize and obtain biogas having an increased percentage of supplied dry substance since there is practically no need for a supply of water from, for example, the condensate obtained in evaporation or drying. Because of the low energy requirement when using a dry fermenter for evaporation and drying the digestate the percentage of top dressing transposed to the base dressing accordingly increases to 50% and more. In other words, the branch flows of, for one thing, the base dressing and, for another, of the top dressing resulting from mechanical conditioning can be reunited in the main.

In addition to using the base dressing stemming from drying as a high-quality agricultural fertilizer, recycling the base dressing by gasification can be provided to produce a high-energy gas. Gasification will now be explained in the scope of describing a “second recycling mode” of the invention.

Following the basic recycling mode as described above for upgrading a low-quality biomass into organic fertilizers is in accordance with an advantageous embodiment of the invention an additional second recycling mode as described in the following to which the same as that commented above as regards the basic recycling mode applies accordingly:

In the second recycling mode in accordance with the invention the dry base dressing fraction is pelletized in a pellet press, although as an alternative an extruder can be employed. This second recycling mode is particularly of advantage when the organic fertilizer produced has an excess which cannot be recycled as a dressing. In accordance with the invention the resulting organic fertilizer is upgraded as a recyclable crude by obtaining, instead of the original source of energy, for example, a new source of energy in the form of pellets for further use. The energy needed in this case for pelletizing can be taken from the energy made available by the biothermal power plant. To take into account that upgrading the original digestate is to be achieved by recycling the energy generated by the biogas in meeting the energy requirement, it is expedient to include a dry fermenter for generating the biogas in subsequent pelletizing.

Thus, from pellizing itself or the dry output of the conditioner, a new source of energy is produced which as described in the following in accordance with the so-called second recycling mode can be combustioned and/or gasified. Recycling the dry output in this way is implemented to advantage immediately following the drying or pelletizing process, i.e. in the immediate vicinity of the biogas plant to feed the resulting energy into the electricity grid of the biothermal power plant and to admix the gas obtained from gasification with the biogas. However, as an alternative, it is just as possible to provided for a decentralized arrangement in combustioning or gasifying the gas to supply commerce with the resulting pellets in the form of wood pellets or admix them in such.

For the purpose of generating energy a steam turbine can be interposed in pellet combustion which in turn operates a generator which then outputs the resulting electricity into the grid of the biothermal power plant. Analogously, a gas turbine or a piston engine can be interposed in gasification of the pellets or dry product of the conditioner which likewise outputs electricity into the grid of the basic biothermal power plant via generator operation. The residuals resulting from combustion or gasification, i.e. on the one hand, ash (combustion) and carbon (gasification) can in turn be employed as fertilizer. For best results a multi-gasifier can be provided for gasification which can be fed with a variety of materials for gasification. The system can thus also be adapted to accommodate fluctuations in the crude yield by, for example, adding wood to compensate a poor maize yield, for lack of rain, at least in part in thus ensuring a continuing supply of energy.

In accordance with still another advantageous embodiment—described in the following by the term “total recycling mode”—that as commented above as regards the basic recycling mode applies analogously to the total recycling mode:

In accordance with this embodiment recycling is focussed on the dry product obtained from the organic fertilizer in gasification, the resulting gas of which is added in accordance with the invention to the biogas so that electricity can be generated from this mixture in the biothermal power plant, making it possible to tweak the energy balance in the direction of a higher energy yield. Among other things, this is due to the efficiency “□” in obtaining energy from the biogas/gas mixture in the biothermal power plant being higher in each case than the efficiency in single recycling, because of how the gases favorably supplement each other, as tests have shown.

The same as for the “second recycling mode” using a common conditioner for the cake of the digestate as for the concentrate from the evaporation is of advantage for the “total recycling mode” too in permitting an increase in the mass flow of top dressing into the base dressing—as described above for the example of the “second recycling mode”—as high as 100%, especially in generating additional electricity. Thus, roughly 35% more electricity can be gained for the same land or renewable crudes rating which in view of the anticipated crudes shortage is decisive.

The gas produced in gasification still includes components, such as, for example, mainly nitrous compounds, which are detrimental to further recycling of the gas, resulting in Nox and compounds thereof when recycling the gas. To eliminate these noxious fractions the gas is cleaned by passing it through the liquid fraction of the digestate produced in concentration at least in part to advantage. Another partial flow of the gas can also be directed through the concentrate or condensate for its cleaning. Tests in this respect have shown that the components detrimental to gas recycling are removed. The cleaned gas can then be made available either by a gas turbine or piston engine for generating electricity or it is added to advantage to the gas obtained from fermenting.

Since the nitrous fractions of the gas obtained from gasification of the base dressing or pellets are returned by the removal process to the liquid fraction of the digestate or to the concentrate from evaporation, the latter is reenriched with precisely these removed components, expediently resulting in upgrading a low quality dressing into an organic fertilizer. When the gas is directed through the condensate it can likewise be directly admixed in the flow of the dressing.

In one variant of the method in accordance with the invention the gas resulting from gasification can also be cleaned by directing it through the condensate resulting from evaporation, at least from time to time. Doing this makes sense when the mass flow from top dressing to the base dressing is practically 100% and, at the same time, the resulting base dressing is to be supplied exclusively for gasification, at least for a certain period of time. Scrubbing the gas in the condensate finalizes filtering off the components detrimental to recycling the gas in preventing them from “running in circles” as in the concentrate which is redried in the transposed flow to the base dressing and then subjected to gasification.

In other words, the invention now makes it possible in the scope of recycling the base dressing by means of gasification to generate gas in two stages from biogas fermentation and optimized gasification of the digestate in two gas flows, each independent of the other, i.e. it now making it possible to maximize the energy yield whilst simultaneously enhancing the concentration of the recyclables from the original starting substrate. This combination adds to the flexibility of the method as to the requirement for more energy or more fertilizer.

To finish with, it is to be noted that the gasification residuals can be enriched likewise with the top dressing or with the condensate partial flow employed in cleaning, here again the resulting product satisfying the requirements on an organic fertilizer so that the gasification residuals can be likewise made use of in agriculture.

### BRIEF DESCRIPTION OF THE DRAWINGS

With reference to the drawings illustrating the basic method of the invention as well as the basic recycling mode, the second recycling mode and the total recycling mode an example embodiment for each will now be described for the case “biogas process”, wherein

FIG. 1 is a flow diagram of the method in accordance with the invention as regards the basic recycling mode;

FIG. 2 is a flow diagram of the method in accordance with the invention as regards the so-called second recycling mode;

FIG. 3 is a flow diagram of the method in accordance with the invention as regards the so-called total recycling mode;

FIG. 4 is a diagrammatic view of an advantageous belt drying system in accordance with the invention.

### DETAILED DESCRIPTION

The flows as shown in the FIGs. are symbolized as follows:

| | |
|---|---|
| | mass flow |
| ----------- | gas |
| ........... | thermal energy |
| ooooooooooo | alternative heat |

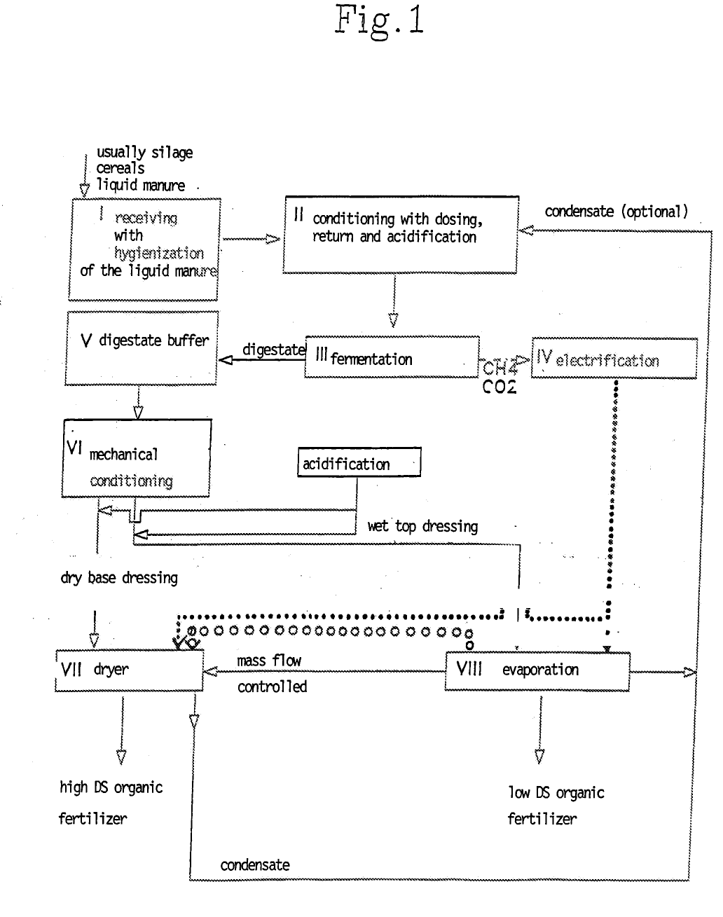

Referring now to FIG. 1 there is illustrated in a diagrammatic flow diagram how maize silage, cereals and/or liquid manure or the like is furnished to a receiving station (I) for hygienizing the liquid manure. After this, this biological starting material is recycled including dosing, return (II) and, as a rule, also including preacidification. Preacidification serves to digest complex carbon compounds since the fermentation bacteria of the subsequent fermentation(III) preferably deplete only simple carbon compounds. Preacidification usually requires attaining a pH of 6.0 or lower. The residence time of the contaminated substance input in preacidification depends on how long it takes to digest the individual components. Thus, for instance, press remainders such as solid peel residuals require a longer residence as compared to organically loaded fluid. For differing residence times a retention system to hold back components problematic in digestion is thus expedient as it reads from the patent application EP 1 419 995 of the present applicant. Were solids lacking sufficient preacidification also to be made available, their subsequent acidification in the main fermenter would ruin the methanization process.

It is furthermore of advantage to allow the starting substrate, especially maize or other silages in mashing, to rest, where necessary lightly stirred, to allow the air to escape. The advantage of this is that it prevents layers of scum forming in subsequent fermentation (III) in optimizing runoff of NH4 as formed in processing the digestate (as explained above). Receiving (I) and conditioning (II) may also include a sand trap.

Once the resulting crude mix/substrate is sufficiently acidified it is forwarded to fermentation (III) without the risk of any further acidification occurring in the anaerobic fermenter which would be to the disadvantage of the fermentation process because of damage to the methane bacteria due to the low pH. In the fermentation step or process the intermediate products of the hydrolysis such as e.g. acetic acid are depleted substantially to methane and carbon dioxide with the aid of acetogenic bacteria and methane bacteria in the anaerobic reactor. The resulting gas mixture is used to obtain thermal energy and electricity in being streamed through a biothermal power plant (IV).

Resulting furthermore from fermentation (III) is a digestate which is directed into a digestate buffer tank (V) which creates a buffer volume for the subsequent mechanical concentration (VI) achieving a continuous infeed for the presses involved in mechanical concentration. As an alternative, concentration may also be done by means of a decanter for separation of solid fractions into liquid fractions by gravity. The dry substance fraction (DS) of the digestate in the digestate buffer amounts to approximately 5 to 7%. The temperature in the digestate buffer is approximately 34° C. As already explained, the digestate is forwarded from the buffer to mechanical presses for concentration thereof.

Two partial flows materialize from mechanical concentration (VI) namely, for one thing, the concentrated cake with an elevated dry substance content of approximately 25%, this partial flow being termed base dressing, and, for another, the liquid fraction obtained from pressing with a lower dry substance content of approximately 3% termed top dressing in the following. Inhibiting frothing in these flows can be done as already described. The top dressing is subsequently filtered in vibrating sieves to hold back fibrous fines which could clog up subsequent evaporation or reverse osmosis with ultrafiltration. Sieving the top dressing results in a solids fraction as a concentration with a dry substance fraction of approximately 10% which after concentration e.g. by means of a decanter can also be forwarded to the dryer VII.

An acid, e.g. sulfuric acid, is added to both partial flows, i.e. the concentrated cake and the liquid top dressing especially because of the subsequent evaporation of the top dressing or optional drying the press cake to permit binding the nitrous fractions contained in the digestate in the form of ammonia (NH4); resulting in the pH being shifted into the acidic domain, adapted to the temperature conditions, as a rule ≦pH 6. This shift in the pH creates frothing in the liquid part, inhibiting frothing in these flows being possible as described above. No froth forms in the cake in accordance with the invention.

The liquid fraction coming from the concentration is then subjected to vacuum evaporation, resulting in an aqueous condensate and a concentrate with approximately 12 to 18% dry substance. In evaporation the top dressing flow is initially preheated from an originally temperature of 29° C. to approximately 63° C. Subsequent evaporation is done in several vacuum stages in making use of the heat given off by the biothermal power plant.

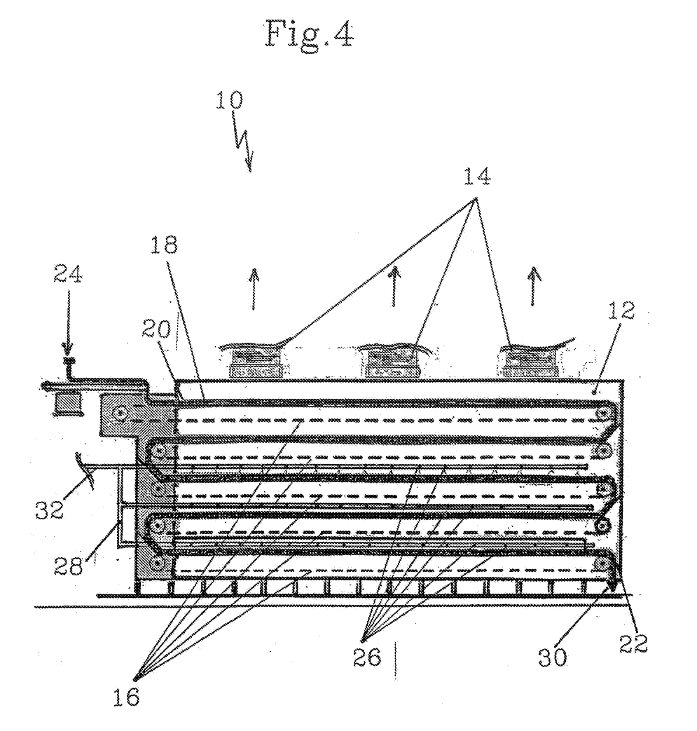

As regards the partial flow of the concentrated cake, this is subjected to drying (VII). Conditioning occurs, for example, in a belt dryer in which the concentrated press cake is forwarded on a horizontal stack of belts so that the cake, beginning at the topmost belt, after completing the run thereon, drops onto the belt below and is zig-zagged down to the bottommost belt. Details as to the drying process now follow with reference to FIG. 4. To dry the base dressing in the belt dryer a heat exchanger is provided with which the heat given off from the biothermal power plant is made use of. The waste heat of the source of energy of the biothermal power plant has a temperature of approximately 145° C. when entering the belt dryer, dropping to approximately 120° C. at the output. Drying the press cake is thus achieved exclusively with the waste heat energy available in the biothermal power plant. The condensate resulting from air cooler the dryer is buffered and subsequently returned, for example, to the mash in the fermenter. The dried cake coming from the dryer is termed base dressing irrespective of its content and composition as well as what it is intended for. Instead of the biothermal power plant being the basic energy supply the last stage in condensation of evaporation may also furnish the energy. This is particularly an advantage when the fermentation system generates only feed gas, making energy “short”, it then, whenever this is the case, being good practice to fully or partly combustion the organic fertilizer to generate energy.

Whilst the top dressing has a nitrogen content of approximately 6%, that of the dried base dressing is as a rule less than 3%. So as to upgrade the base dressing into a top quality, i.e. purely organic fertilizer, part of the top dressing concentrate is forwarded in a controlled mass flow to the base dressing in the belt dryer. This controlled mass flow depends, among other things, on the energy available by the biothermal power plant when the system is to run on its own energy. For, the available waste heat is used for drying results in the amount of waste heat being a factor influencing the dryable mass and thus also the wet fraction available to be added to the base dressing. Thus, in this case the mass flow can constitute approximately 20% of the top dressing produced in all, resulting in the combination of both partial flows—i.e. top dressing on the one hand and base dressing on the other—a high quality organic fertilizer being produced as a solid, the nitrogen fraction of which tops a 3% limit. Transposing top dressing into the base dressing may, in addition to the cited example for nitrogen enrichment of the base dressing, likewise relate to the potassium or phosphorous fraction.

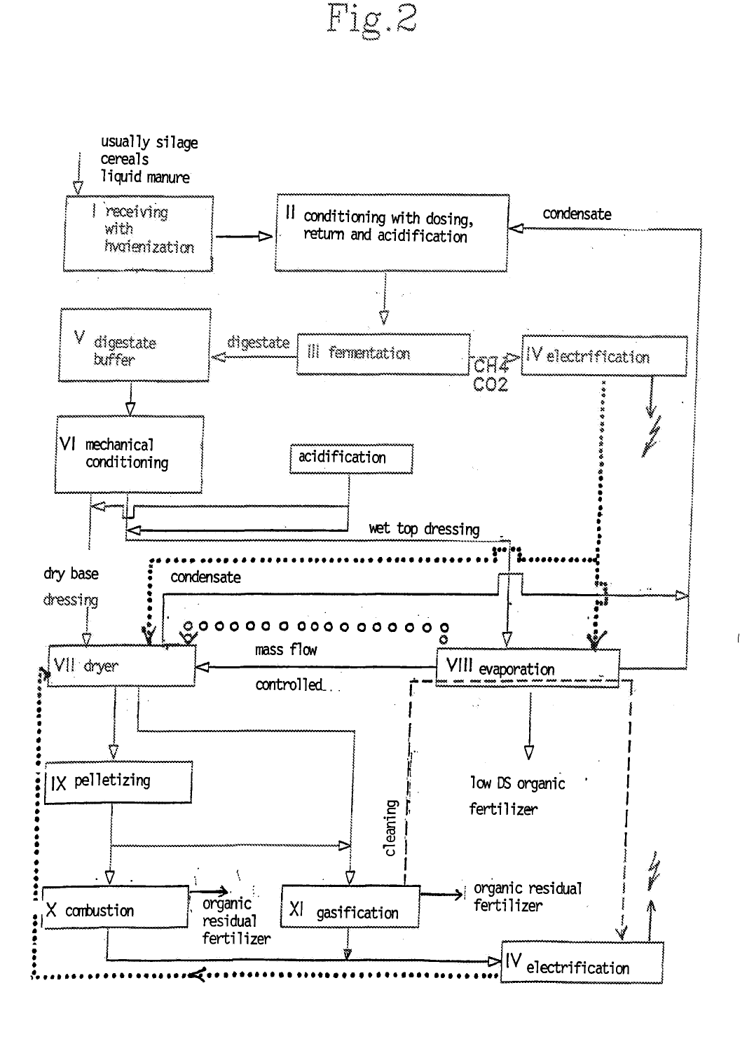

Referring now to FIG. 2 there is illustrated diagrammatically the so-called second recycling mode as a sophistication over the basic recycling mode as just described. In accordance with the second recycling mode the substrate stemming from drying (VII) is processed further by subjecting it to pelletizing (IX) and/or forwarding it to subsequent gasification (XI) or combustion (X), a pelletizer or an extruder being possibly employed as the device for compacting the dry product.

The substrate stemming from the dryer has a dry substance percentage of approximately 80% on an average. The compressed pellets stemming from pelletizing (IX) having a dry substance percentage of approximately 85% are forwarded to combustion and/or gasification to generate electricity by means of a fuel cell, combustion boiler, piston engine or gas turbine for e.g. electrification (IV) of the biothermal power plant. Upgrading the dry substance stemming from the dryer in quantity with the aid of part of the base dressing stemming from evaporation or condensate partial flow thus results in further optimization in managing the process in recycling residuals stemming from fermentation.

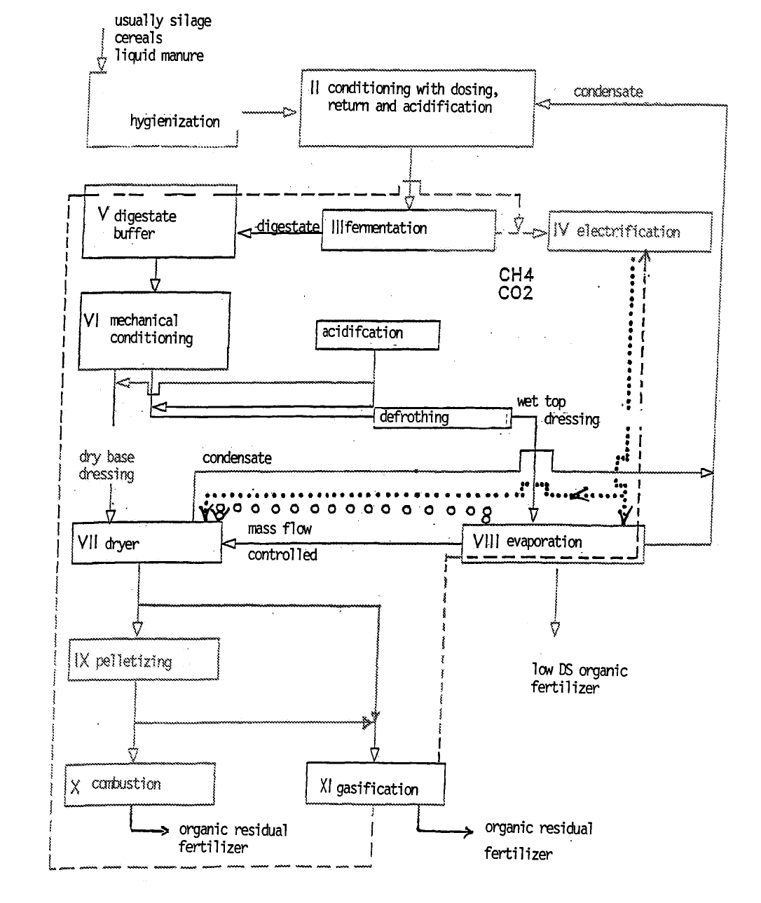

Referring now to FIG. 3 there is illustrated diagrammatically the basic principle of the so-called “total recycling mode” signifying a sophistication over the second recycling mode as shown in FIG. 2 and the basic recycling mode as shown in FIG. 1. In accordance with the total recycling mode the gas stemming from the gasification (XI) of the dry substance is mixed with the gas stemming from fermentation and forwarded together for electrification (IV) of the biothermal power plant.

To advantage the gas stemming from gasification of the dry substance is guided for cleaning through the digestate in the digestate buffer tank (V). As an alternative the gas can be cleaned also by directing it through the concentrate of the top dressing from the evaporation or through the condensate or as brought out from a partial flow. In other words, the gas obtained from gasification is upgraded both energywise and ecologically when it is passed through, for example, the liquid fraction of the wet flow resulting from concentration of the digestate. This liquid fraction is likewise fortified which although to the disadvantage in recycling the gas, cleaning the gas also constitutes a high quality means of upgrading the liquid fraction since it is enriched with substances suitable for utilization as an organic agricultural fertilizer. Passing the gas through the condensate should only be intended, however, when the condensate is no longer needed, for example, as water for mashing the silage on entering the fermenter. The positively charged flows may also be added to the organic residual (not shown). Should the condensate still contain nutrients or other substances (for instance salt) reverse osmosis can be employed so as not to complicate mashing and the subsequent stages.

Transposing the gas stemming from gasification of the dry substance optimizes the “production costs” of the system (IV), and energy yield is increased due to the higher efficiency in recycling the mixed gas. Whilst the efficiency of a gas turbine in gasification of the dry substance stemming from drying (VII) approximates 25%, the efficiency □□ in recycling the gas mixture from fermentation and gasification is as high as 44%. In all, however, this constitutes total recycling of the dry substances resulting with the digestate since in total electrification in (IV) so much waste heat materializes that all fertilizer dressings resulting from evaporation (VIII) or recyclables for drying (VII) can be handled.

With the above recycling modes, namely basic recycling mode, second recycling mode and total recycling mode with optimized gasification, the system as a whole now features a flexibility permitting selection of the product or energy as desired in the end, i.e. purely organic fertilizer, energy in the form of a digestate or pellets or electricity. This flexibility now also makes it possible to optimally adapt the system as a whole technically to the available starting material. Thus, should it happen that the maize yield is insufficient for fuelling the system, the system can now be run on e.g. green cuttings or wood for gasification in the multi-gasifier. Being able to adapt the system to fluctations in available crudes and energy demand likewise enables the system to be adapted to meet differing site requirements. In other words, the processing flexibility created for the system now makes it possible to expediently employ the system anywhere geographically.

In all of the cases described a cooling unit and a heating unit are assigned to the dryer (VII)—evaporator (VIII) systems (not shown) to, e.g. as a controller, purely compensate fluctuations in the energy requirement on startup and to harmonize changes in performance among other things with the energy produced by the biothermal power plant at the time.

Referring now to FIG. 4 there is illustrated diagrammatically a belt dryer as may be used to advantage for drying the base dressing coming from the digestate concentrator and a top dressing resulting from evaporation, it being important that the dryer is not only intended as usual “for storeability” but also promoting an optimum residual moisture content for all subsequent steps in the method such as pelletizing or gasification.

The conditioner 10 features a drying space 12 through which the material is conveyed from a starting point 20 of the drying space to an end point 22 thereof. Conditioning itself is done by means of exhaust air drawn through the drying space 12 to absorb the wet fraction and discharging it. Connecting the exhaust air outputs 14 as shown in FIG. 4 are the exhaust air pumps (not shown). The thermal energy required for drying is taken as a rule from the waste heat of the biothermal power plant but may also be made available in addition by heaters (not shown) of the conditioner or e.g. also from the final stage of the evaporation. The belt dryer may also feature a filter unit (not shown) for dust extraction.

The material to be dried consists of a first wet fraction, namely the base dressing coming from the mechanical concentrator and having a dry substance percentage of around 75%, and a second wet fraction, the so-called top dressing from the evaporation having a dry substance percentage of approximately 15%.

As regards how the base dressing is conveyed:

The first wet fraction, i.e. the base dressing, is forwarded to a first wet fraction input 24 of the belt dryer. At this location of the wet fraction input 24 a distribution rake (not shown) may be provided. The first wet fraction is conveyed on a topmost conveyor belt 16 lengthwise through the drying space 12 horizontal until at the end of the conveyor belt the wet fraction drops via a chute (not shown in detail) onto the next belt below. Transferring the wet fraction in this way results in it being thoroughly mixed at the transfer locations, homogenizing the moisture distribution in the first wet fraction. The conveyance path 18 of the first wet fraction is thus from the starting point 20 to the end point 22 of the drying space 12, this conveyance path 18 being illustrated in FIG. 4 by the solid line. At the end point 22 of the drying space the dry material production is output at the product discharge 30. As shown, the belt dryer has a stack of five conveyor belts one above the other. Depending on the first and second wet fraction to be dried, more or fewer conveyor belts may be provided. The speed too, of each belt can be separately continuously controlled, for example, by a frequency converter. The conditioner can thus be optimally adapted to the product needing to be dried. The conveyor belts 16 may also be sieve belts of differing mesh.

As regards how the second wet fraction is conveyed:

The first wet fraction, i.e. the base dressing, on having half passed through the drying space 12, is “rained” upon by a second wet fraction, i.e. the top dressing which is ported into the drying space 12 via a second feeder 32 for the second wet fraction. Because the dry substance fraction of the top dressing is so low that it can be piped as a fluid, the top dressing is conveyed into the drying space 12 through pipes 28.

Provided in the horizontal stack of pipes 28 as shown in the FIG. Is a multiplicity of ports for the second wet fraction input 26 by means of which the top dressing is applied to the first wet fraction conveyed by the each belt. How much top dressing is discharged by each second wet fraction input 26, i.e. each output nozzle, can be controlled to advantage by means of valves. At the end point 22 of the drying space 12 the dry product consisting of dried base dressing and dried top dressing is made available at the product discharge 30, this dried mixture having a dry substance percentage of approximately 80%. This dried product can be utilized either as an agricultural fertilizer dressing or further processed to advantage, for example by subjecting it to gasification, to produce energy in the form of a gas which can be admixed with the biogas steamming from fermentation.

In conclusion it is to be noted that drying the base dressing in the conditioner in accordance with the invention is also possible without the addition of the top dressing.

1-40. (canceled)

41. A method for processing organic residuals materializing in a fermenter and subjected to mechanical conditioning comprising at least one concentration step, wherein subsequent to mechanical conditioning a resulting digestate liquid flow therefrom is subjected to treatment for the production of a concentrated top fertilizer dressing, on the one hand, and, on the other a condensate, and the digestate cake stemming from concentration is run through a dryer for the production of a base fertilizer dressing and wherein for its enrichment with energy recyclable materials the partial flow of the base dressing production receives a controllable fraction of the top dressing.

42. The method as set forth in claim 41, characterized in that the waste heat energy of a biothermal power plant operated with the biogas is used to dry the base dressing and the controllable mass of the transposed top dressing concentrate is determined by the available waste heat.

43. The method as set forth in claim 41, characterized in that the treatment for production of a concentrated top dressing involves an evaporation or ultrafiltration with reverse osmosis, the top dressing representing the concentrate of the evaporation or the retentate from the ultrafiltration with reverse osmosis.

44. The method as set forth in claim 1, characterized in that the top dressing is added to the base dressing during its drying.

45. The method as set forth in claim 44, characterized in that the concentrated digestate is dried using a belt dryer system and admixing the controlled mass flow of top dressing into the base dressing is done in substantially half the throughflow of the digestate cake through the belt dryer system by raining the base dressing with the top dressing.

46. The method as set forth in claim 41, characterized in that the controllable transposition of the mass flow is designed so that at least the nitrogen and/or potassium and/or phosphorous fraction or compounds thereof in the base dressing mixture amounts to at least 3%.

47. The method as set forth in claim 41, characterized in that the base dressing coming from drying is gasified.

48. The method as set forth in claim 47, characterized in that the gas materializing from gasification is cleaned by passing it through the concentrate or condensate resulting from evaporation.

49. The method as set forth in claim 47, characterized in that the gas obtained from gasification is admixed with the biogas obtained from fermentation.

50. A device for recycling biomass containing interconnected:
a) a fermentation tank and means for generating a biogas,
b) means for separating a digestate from the fermentation tank,
c) means for concentrating the digestate, the concentration forming a cake as well as a liquid fraction,
d) means for separating a concentrate from the liquid fraction, as well as
e) means for conditioning the concentrated cake, and
f) means for a controllable transposition of parts of the resulting concentrate into the conditioned cake.

51. The device as set forth in claim 50, characterized in that a buffer for receiving the digestate coming from fermentation is provided.

52. The device as set forth in claim 50, characterized in that the means c) for concentrating the digestate comprise a screw press separator.

53. The device as set forth in claim 50, characterized in that the means c) for concentrating the digestate comprise a decanter.

54. The device as set forth in claim 50, characterized in that the means d) for separating a concentrate comprise an evaporator.

55. Use of a device for recycling biomass containing interconnected:
a) a fermentation tank and means for generating a biogas,
b) means for separating a digestate from the fermentation tank,
c) means for concentrating the digestate, the concentration forming a cake as well as a liquid fraction,
d) means for separating a concentrate from the liquid fraction, as well as
e) means for conditioning the concentrated cake, and
f) means for a controllable transposition of parts of the resulting concentrate into the conditioned cake.
for implementing a method for processing organic residuals materializing in a fermenter and subjected to mechanical conditioning comprising at least one concentration step, wherein subsequent to mechanical conditioning a resulting digestate liquid flow therefrom is subjected to treatment for the production of a concentrated top fertilizer dressing, on the one hand, and, on the other a condensate, and the digestate cake stemming from concentration is run through a dryer for the production of a base fertilizer dressing and wherein for its enrichment with energy recyclable materials the partial flow of the base dressing production receives a controllable fraction of the top dressing, for generating electricity and/or heat or for the production of fertilizer dressings.
